(19) United States
(12) Patent Application Publication
(10) Pub. No.: US 2016/0287241 A1
(43) Pub. Date: Oct. 6, 2016

(54) MEASUREMENT RING AND MONITORING

(71) Applicant: CAE HEALTHCARE CANADA, Saint-Laurent (CA)

(72) Inventor: Hugo Azevedo, Dollard-des-Ormeaux (CA)

(21) Appl. No.: 14/673,912

(22) Filed: Mar. 31, 2015

(51) Int. Cl. A61B 17/02; A61B 5/107; G09B 23/28; A61B 5/11

(52) U.S. Cl. CPC A61B 17/0293; A61B 5/11; A61B 5/1076; G09B 23/285

(57) ABSTRACT

The present disclosure relates to a measurement ring for positioning at an orifice of a patient. The measurement ring comprises a hollow channel, an annular lip, at least one sensor and a communication unit. The annular lip is located at a first extremity of the hollow channel. The annular lip and hollow channel form an insertion channel for inserting at least one instrument in the orifice of the patient. The at least one sensor measures at least one parameter related to insertion of the instrument in the insertion channel and generate insertion data. The communication module transmits the insertion data to a monitoring apparatus.

### TECHNICAL FIELD

The present disclosure relates to the field of medical procedures. More specifically, the present disclosure relates to a measurement ring to be used in an orifice of a patient when performing a medical procedure so as to monitor the ongoing procedure.

### BACKGROUND

Before performing medical procedures, medical professionals require training. In the past, the training was performed on patients under the supervision of experienced medical professionals. However, training on patients is risky as inadequate or inappropriate movements or procedures may cause serious damages. To overcome these problems, some hospitals and medicine schools are gradually offering training on mannequin simulators. Mannequin simulators mimic anatomical characteristics of patients, and are used to simulate medical procedures.

To enhance and improve simulated medical procedures, it is necessary to obtain measurements and data of undergoing procedures, so as to have complete information for creating realistic and detailed simulations. To date, there is very limited information and measurements obtained from medical procedures including inserting probes or surgical equipment through an orifice of a patient. There is therefore a need for a new device to measure parameters of undergoing medical procedures performed through an orifice of a patient.

### SUMMARY

In accordance with a first aspect, the present specification relates to a measurement ring for positioning at an orifice of a patient. The measurement ring comprising a hollow channel, an annular lip, at least one sensor, a communication module. The annular lip is positioned at a first extremity of the hollow channel. The annular lip and hollow channel form an insertion channel for inserting at least one instrument in the orifice of the patient. The at least one sensor is adapted for measuring at least one parameter related to insertion of the instrument in the insertion channel and generate insertion data therefor. The communication module is adapted for transmitting the insertion data to a monitoring apparatus.

In a particular aspect, the present specification relates to a monitoring apparatus for monitoring insertion of at least one instrument through an orifice of a patient. The monitoring apparatus comprises the present measurement ring positioned at an orifice of the patient. The monitoring apparatus further comprises a communication unit for receiving the insertion data transmitted by the communication unit of the measurement ring, and a monitoring engine for monitoring the insertion of the at least one instrument in the orifice of the patient by means of the received insertion data.

### BRIEF DESCRIPTION OF THE DRAWINGS

Embodiments of the disclosure will be described by way of example only with reference to the accompanying drawings, in which:

FIG. 1 is a perspective view of the present measurement ring;

FIG. 2 is a side perspective view of the present measurement ring;

FIG. 3 is perspective view of an alternative of the present measurement ring;

FIGS. 4A-4C are photographs of the present measurement ring inserted into the mouth of a mannequin, with an endoscope inserted therein; and

FIG. 5 is a schematic diagram of a monitoring apparatus.

### DETAILED DESCRIPTION

The foregoing and other features will become more apparent upon reading of the following non-restrictive description of illustrative embodiments thereof, given by way of example only with reference to the accompanying drawings. Like numerals represent like features on the various drawings.

Various aspects of the present disclosure generally address one or more of the problems related to monitoring and measuring parameters related to insertion of instruments (for example endoscope, colonoscope, bronchoscope, etc.) in an orifice of a patient.

The present description relates to a measurement ring, and its use in the field of medical procedures and monitoring thereof. The present measurement ring can be used to train medical professionals in performing procedures in which instruments must be inserted through an orifice of a patient, such as for example the mouth, the nose, ears, or the anus. Furthermore, the present measurement ring and monitoring apparatus can be used during procedures to measure insertion parameters related to the insertion of instruments into the orifice of the patient, and compare the measured insertion parameters with ranges of acceptable measurements. The present measurement ring can also be used with the present monitoring apparatus, so as to collect insertion data and generate therefor procedure results.

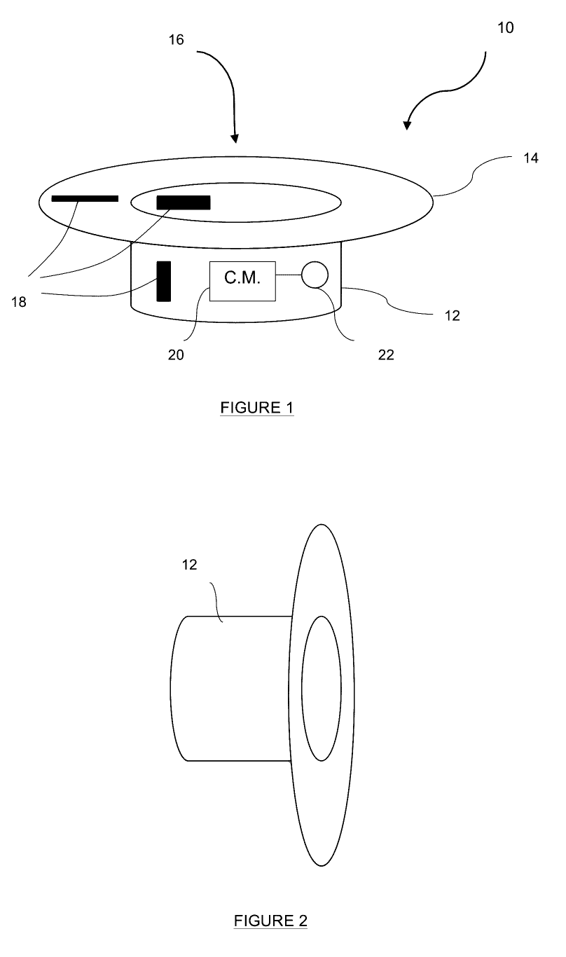

Referring now concurrently to FIGS. 1 and 2, there is shown a perspective view and a side elevation view of the present measurement ring 10. The measurement ring 10 comprises a hollow channel 12 and an annular lip 14 at an extremity of the hollow channel 12. The annular lip 14 and the hollow channel 12 define an insertion channel 16 for instruments during a medical procedure. The hollow channel 12 is shaped, sized and proportioned for smooth and comfortable insertion into an orifice of a patient or mannequin. Hence, depending on the application, the hollow channel will be sized smaller for use in the nose and ears, and larger and longer for use in the mouth. The hollow channel 12 can be cylindrical, cone-shaped with a truncated end, etc. The hollow channel 12 may be made of a solid material or a semi rigid material. Soft materials could also be used to manufacture the hollow channel 12 for applications in which the hollow channel 12 is flexible upon its length to allow smooth insertion into the orifice while the instrument is inserted there through.

The annular lip 14 is shaped, sized and proportioned so as to comfortably remain outside of the body, while allowing the hollow channel 12 to be inserted into the orifice of the patient. For example, if the measurement ring 10 is designed for insertion in the mouth, the annular lip 14 is shaped and sized so as to cover the lips and teeth, while the hollow channel 12 is sized to allow comfortable insertion into the mouth of the patient.

Although shown as en ellipse on FIG. 1, the annular lip could have different shapes and sizes. For example, the annular lip 14 can be symmetrical, asymmetrical, evenly shaped along its internal and/or external periphery, unevenly shaped along its internal and/or external periphery, thinner, thicker, flexible, or solid. The hollow channel 12 and the annular lip 14 may be made as two distinct pieces joined together to form the insertion channel 16, or made as one piece for example by using a mold or 3D printing technology. The measurement ring may be shaped as a mouth block or a bite block. The hollow channel 12 and the annular lip 14 are made of medical grade material, such as for example silicone. With the advance of 3D printing technology, it could also be possible to print in 3D in a material appropriate for use in medical application, a custom-sized hollow channel 12 and annular lip 14 so as to offer maximum comfort to the patient.

The measurement ring 10 further comprises one or several sensors 18. The following description will use the term sensor(s) 18 to concurrently refer to one or several sensors. The sensor(s) 18 is/are positioned on the measurement ring 10 so as to measure any of the following parameters taken solely or in combination: insertion of an instrument in the insertion channel 16, movement of the instrument in the insertion channel 16, position and/or pressure applied by the instrument against the hollow channel 12, speed of insertion of the instrument in the insertion channel 16, pressure of the instrument on the interior of the hollow channel 12 or portion thereof, and pressure of the instrument on the annular lip 14 or portion thereof. Depending on the application for which the measurement ring 10 is manufactured, various types of instruments could be inserted within the insertion channel 16. For example, when the measurement ring 10 is sized and shaped to be used in the mouth of a patient or mannequin, the insertion channel 16 would be sufficiently large to allow insertion of instrument(s) used for medical procedures such for example as intubation and endoscopy.

The sensor(s) 18 may be positioned inside or outside of the measurement ring 10, or within the material of the measurement ring itself. Furthermore, the sensor(s) 18 may be positioned along a portion of the hollow channel 12 and/or along the annular lip 14. Additionally, the sensor(s) 18 may be located along an interior and/or exterior periphery of the hollow channel 12 or annular lip 14, or along a portion of the length of the hollow channel 12 and/or of the annular lip 14. The sensor(s) 18 may be positioned concurrently on the hollow channel 12 and the annular lip 14. Examples of positioning of the sensor(s) 18 are shown on FIG. 1 for exemplary purposes only. The sensor(s) 18 are positioned so as to allow measurement of insertion parameters of an instrument (not shown) through the insertion channel 16 and generate corresponding insertion data.

The sensor(s) 18 may consist of any of the following types of sensors, taken singly or in combination: pressure sensor(s), position sensor(s), movement sensor(s), tensile sensor(s) and distance sensor(s). The sensor(s) 18 measure(s) parameter(s) related to the insertion of one or several instruments in the insertion channel 16 independently of the instrument being inserted, i.e. without requiring any modification to the instrument currently used.

In the event that the sensor(s) 18 is/are pressure sensor(s), the insertion data comprises pressure applied by the instrument on the sensor(s) 18. When the sensor(s) 18 is/are position sensor(s), the insertion data comprises a position and/or angle and/or rotation and/or depth and/or proximity of the instrument(s) being inserted within the insertion channel 16 of the measurement ring 10 or where/how the position sensor(s) is/are located precisely. When the sensor(s) 18 is/are movement sensor(s), the insertion data generated corresponds to movement of the instrument(s) in the insertion channel 16 or on the measurement ring 10. When the sensor(s) 18 is/are tensile sensor(s), the insertion data generated corresponds to the tensile movement of the material of the measurement ring 10 caused by the insertion of the instrument(s), measured along the measurement ring 10. When the sensor(s) 18 is/are distance sensor(s), the insertion data generated corresponds to a distance of the instrument(s) being inserted with respect to the distance sensor.

By using one or a plurality of sensor(s) 18 on the measurement ring 10, it is possible to measure various parameters quantifying and qualifying how the insertion of instrument(s) is performed by a medical professional. The sensor(s) 18 may all be functioning concurrently, in series, in a predetermined sequence, in a random sequence or on demand. For example, the sensor(s) 18 may continuously take measurements but only start generating insertion data once a predetermined threshold measurement value is reached, on a per sensor basis.

The measurement ring 10 further comprises a communication module 20. The communication module 20 receives the insertion data generated by the sensor(s) 18, and generates therefor measurement signal(s). The measurement signal comprises the insertion data generated by the sensor(s), with an identification of the corresponding sensor. The communication module 20 thus comprises a processor and a memory (not shown for clarity purposes). The communication module 20 generates a measurement signal corresponding to any standard or proprietary protocol, such as for example WiFi, Bluetooth, or any other appropriate communication protocol. The communication module 20 further comprises an antenna and a transceiver (not shown for clarity purposes) for transmitting the measurement signal. Although shown on the Figures as positioned on the hollow channel 12 of the measurement ring 10, the communication module 20 could conversely be located on the annular lip 14. Additionally, the measurement ring 10 could comprise two communication modules 20, one communication module 20 on the hollow channel 12 for communicating the insertion data generated by the sensor(s) 18 located on the hollow channel 12, and another communication module 20 on the annular lip 14 for communicating the insertion data generated by the sensor(s) 18 located on the annular lip 14. In another alternative, there could be one communication module 18 per sensor 18, co-located therewith.

The communication module 20 may further receive command signals from a separate entity such as a monitoring apparatus (discussed further). The command signals comprise an identification of the sensor(s) from which a measurement is requested.

The measurement ring 10 further comprises a power source 22, such as for example a battery or a power cord plugged into an electric outlet or another electronic device (not shown). Alternatively, the power source 22 could consist of electrical contacts between the measurement ring 10 and a mannequin to power the measurement ring 10. The power source 22 powers the sensor(s) 18 and the communication module 20. Electrical connections between the power source 22 and the sensor(s) 18 and the communication module 20 are embedded within the material of the measurement ring 10 so as to avoid any electric shock with the instrument(s) inserted within the insertion channel 16, the patient and the medical professional. The electric connections are not shown on the Figures for clarity purposes, but any type of material and technique known in the field of medical devices and implants could be used to electrically connect the power source 22 to the sensor(s) 18 and communication module 20.

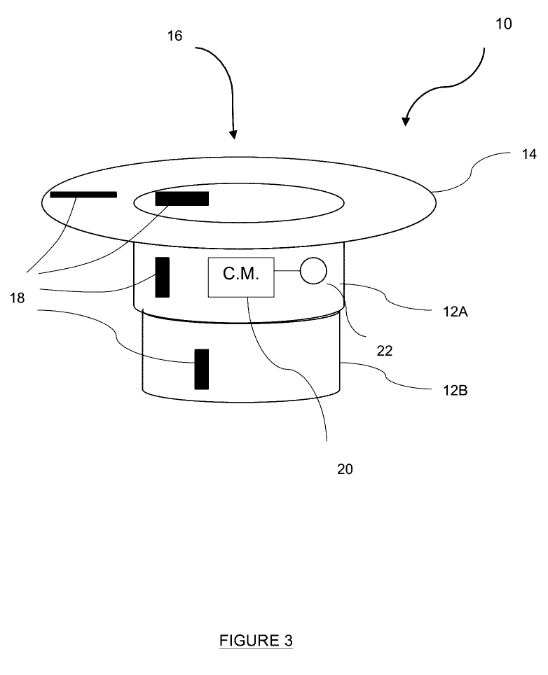

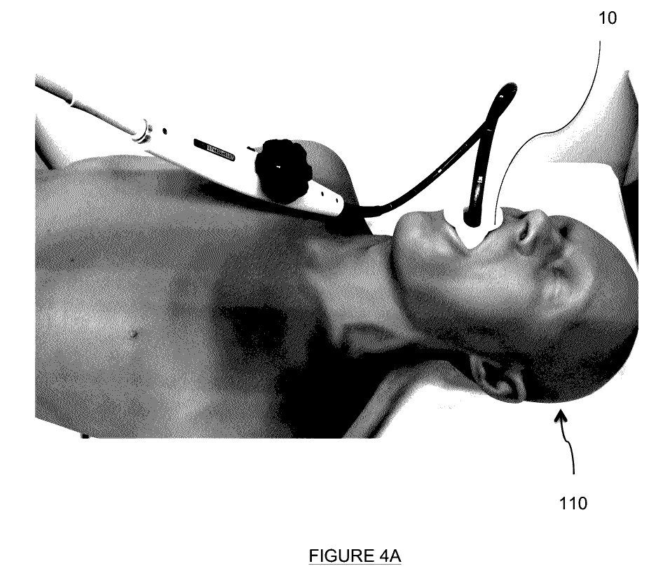

In another particular aspect shown on FIG. 3, the hollow channel 12 of the measurement ring 10 is composed of a series of telescopic hollow channel segments 12A and 12B. The hollow channel segments 12A and 12B are connected at an end of the first telescopic hollow channel 12A opposite the annular lip 14. Although only two telescopic hollow channel segments 12A and 12B are shown on FIG. 3, the present measurement ring 10 could accommodate a much greater number of telescopic hollow channel segments. As shown on FIG. 3, the sensor(s) 18 could be positioned on the annular lip 14 and/or on one or several hollow channel segments 12A and 12B. The sensor(s) 18 on the telescopic hollow channel segments 12A and 12B is positioned on the hollow channel in such a manner as it does not prevent expansion or retraction of the hollow channel segments 12A and 12B. When inserted into an orifice of a patient or mannequin, the measurement ring 10 of FIG. 3 could be inserted with the hollow channel segments 12A and 12B grouped together, and upon insertion of instrument(s) within the insertion channel 16, the hollow channel segment 12B could separate from the hollow channel segment 12A into the expanded position. The interior periphery of the hollow channel segment 12B could be narrower than the interior periphery of the hollow channel segment 12A, so as to provide grip along the instrument(s) inserted into its expanded position, as shown on FIG. 3.

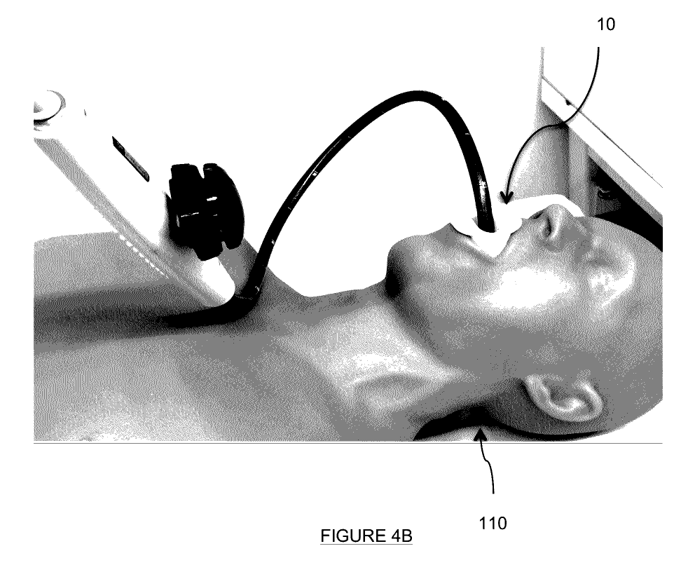

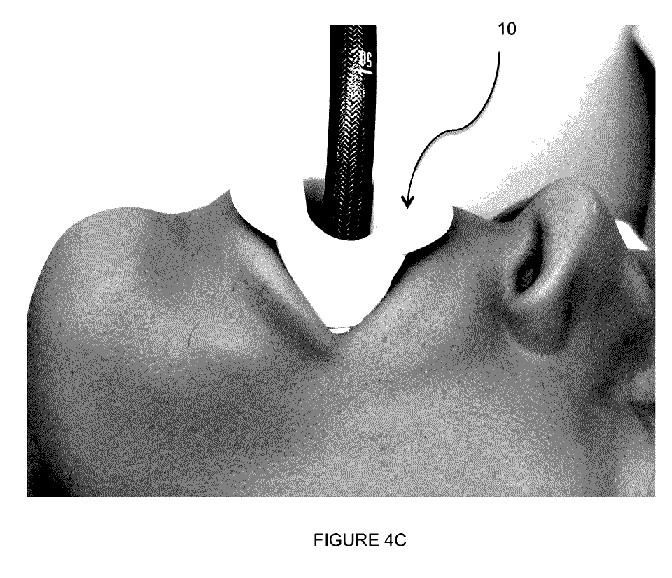

Reference is now made to FIGS. 4A-4C, which are photographs of the present measurement ring 10 inserted in the mouth of a mannequin 110 in place of a patient. As can be appreciated, the annular lip of the measurement ring does not enter the body cavity, and only the hollow channel, or a section thereof, is inserted within the body cavity. In FIGS. 4A-4C, the instrument inserted within the insertion channel 16 of the measurement ring 10 is an endoscope, but any other instrument or plurality of instruments required for performing a medical procedure through the mouth could be inserted within the insertion channel of the measurement ring.

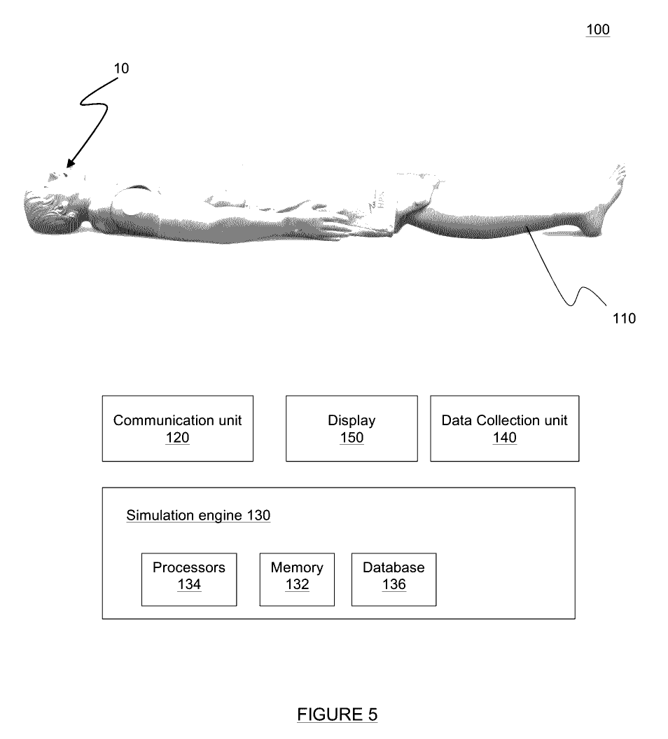

Reference is now made concurrently to FIGS. 1 to 5 where FIG. 5 is a schematic functional diagram of the present monitoring apparatus 100. The monitoring apparatus 100 comprises the measurement ring 10, to be inserted into an orifice (on FIG. 4 the mouth) of a patient 110. As the measurement ring 10 is very small in comparison to the patient 110, the positioning of the measurement ring 10 in the mouth of the patient is shown, but not the measurement ring 10 inside the mouth of the patient 110. As previously discussed, the measurement ring 10 could be used on any orifice of the patient 110, but the following description will refer to the mouth for simplicity purposes.

The monitoring apparatus 100 further comprises a communication unit 120 for wirelessly communicating with the communication module 20 of the measurement ring 10. The communication unit 120 of the monitoring apparatus and the communication module 20 of the measurement ring 10 may wirelessly communicate on an ongoing basis, on a per demand basis, or when a predetermined value measured by one of the sensor(s) of the measurement ring 10 is reached. The communication unit 120 of the monitoring apparatus 100 and the communication module 20 of the measurement ring 10 communicate using any known wireless protocol, either standard or proprietary.

Alternatively, the communication unit 120 of the monitoring apparatus 100 and the communication module 20 of the measurement ring 10 may be adapted to communicate via a physical connection using any known protocol, either standard or proprietary.

The communication unit 120 of the monitoring apparatus 100 is connected to a monitoring engine 130. The monitoring engine 130 comprises instructions stored in memory 132 to be executed by one or several processors 134. The memory 132 may consist of RAM, ROM, FlashDrive, memory banks, or any other type of memory either alone or in combination known in the industry. The instructions stored in the memory 132 may have been coded and compiled using any type or programming software known in the art, so as to produce an executable set of instructions stored in memory 132.

The executable set of instructions stored in memory 132 is executed by the processors 134. The processors may consist of a single processor or multiple processors either in series and/or parallel. The executable set of instructions, when executed, generates the monitoring engine 130 and the interactions of the monitoring engine 130 with the measurement ring 10 through the communication unit 120 of the monitoring apparatus 100 and the communication module 20 of the measurement ring 10. The processors 134 further retrieve and store monitoring related data in a database 136. The monitoring related data comprises both the data required to monitor a procedure, but also the data collected during the procedure. The data collected by the monitoring apparatus 100 is received either directly by the data collection unit 140 or through the communication unit 120 of the monitoring apparatus 100. The data collection unit 140 extracts the insertion data communicated by the communication module 20 of the measurement ring 10 and provides the insertion data to the processors 134. The processors 134 use the insertion data in the execution of the instructions so as to provide the insertion data to the monitoring engine 130. The processors 134 further store the insertion data in the database 136 for future reference such as when evaluating performance of a medical professional after a procedure or for creating simulation data to be used by a patient simulator used with the measurement ring 10.

The communication unit 120, the simulation engine 130 and the data collection unit 140 can be collocated (e.g. housing) or accessible remotely (e.g. via a local or distant network, training facility, etc.).

The monitoring apparatus 100 further comprises a display 150 for displaying at least one of the following: the insertion data received from the communication module 20 of the measurement ring 10, the position/angle/rotation of the at least one instrument in the insertion channel 16 of the measurement ring 10, progression of the insertion of the at least one instrument in the insertion channel 16, pressure of the at least one instrument against the annular lip 14, pressure of the at least one instrument along the interior periphery of the hollow channel 12, relative pressure of the at least one instrument along the insertion channel 16, the position of the at least one instrument along the insertion channel 16, acceptable measurements for any of the previously mentioned measurement and positions. The processors 134 may further provide guidelines or information for improving the performance of the medical professional during the medical procedure on the display 150.

The display 150 may further provide an image of an anatomical area. The image of the anatomical area may consist of a simulated ultrasound representation, an augmented reality image, or a photograph of the anatomical area with the position of the instrument inserted through the insertion channel 16 based on the measurements of the sensor(s) 18 and progression of the insertion of one or more instruments. The display 150 could alternately or concurrently display an image of the anatomical area where the instrument(s) would be positioned based on the measurements taken by the sensor(s) 18.

During a medical procedure using the present measurement ring 10, the monitoring engine 130 may further provide instructions to be followed by the medical professional on the display, recommendations, rate the performance of the medical professional while executing the medical procedure, identify to the medical professional during execution of the simulated procedure that some predetermined measurement thresholds have been reached, etc.

By using the present monitoring apparatus 100 while performing a medical procedure with the measurement ring 10, it is thus possible to identify potential problems which may arise, identify the changes or corrections a medical professional must perform when inserting medical instruments in an orifice of a patient, and/or set some standards of good practice for medical procedures including inserting at least one instrument in an orifice of a patient. Other advantages of the present measurement ring 10 used with the monitoring apparatus 100 will become apparent for those skilled in the art of medical procedures simulation.

The present monitoring apparatus 100 may further be used with multiple types of measurement rings 10, for different applications. To ensure maximum accuracy, the monitoring apparatus 100 may further communicate with the measurement ring 10 to determine which type of measurement ring is being used, so as to adapt the monitoring engine 130 accordingly by extracting the corresponding information from the database 136.

Although the present disclosure has been described hereinabove by way of non-restrictive, illustrative embodiments thereof, these embodiments may be modified at will within the scope of the appended claims without departing from the spirit and nature of the present disclosure.

1. A monitoring apparatus for monitoring insertion of at least one instrument in a patient, the monitoring apparatus comprising:
a measurement ring for positioning at an orifice of the patient, the measurement ring comprising:
a hollow channel;
an annular lip at a first extremity of the hollow channel, the annular lip and hollow channel forming an insertion channel for inserting the at least one instrument in the orifice of the patient;
at least one sensor for measuring at least one parameter related to insertion of the instrument in the insertion channel and generating insertion data based on the at least one measured parameter, the at least one measured parameter comprising at least one of a position of the instrument in the insertion channel and a movement of the instrument in the insertion channel; and
a communication module for transmitting the insertion data to a communication unit of the monitoring apparatus;
the communication unit for receiving the insertion data transmitted by the communication module of the measurement ring; and
a monitoring engine for comparing the received insertion data with ranges of acceptable measurements, the received insertion data being representative of at least one of the position of the instrument in the insertion channel and the movement of the instrument in the insertion channel.

2. The measurement ring of claim 1, wherein the hollow channel and the annular lip are made as one piece of medical grade material.

3. The measurement ring of claim 1, wherein the at least one sensor also measures at least one of the following parameters: pressure applied by the instrument against the insertion channel and location point of pressure applied by the instrument against the insertion channel.

4. The measurement ring of claim 1, wherein the hollow channel is collapsible at an end opposite the annular lip.

5. The measurement ring of claim 4, wherein one of the at least one sensor is positioned along the hollow channel in such a manner that the at least one sensor collapses with the hollow channel.

6. The measurement ring of claim 1, wherein one of the at least one sensor further detects pressure applied by the instrument along an internal periphery of the annular lip.

7. The measurement ring of claim 1, wherein one of the at least one sensor is located along a periphery of the hollow channel.

8. The measurement ring of claim 1, wherein one of the at least one sensor is located along a length of the hollow channel.

9. The measurement ring of claim 1, wherein the annular lip is large enough to cover the lips and teeth of the patient when the measurement ring is inserted in the mouth of the patient.

10. The measurement ring of claim 1, wherein one of the at least one sensor is embedded within a material of the hollow channel.

11. The measurement ring of claim 1, wherein one of the at least one sensor is embedded within a material of the annular lip.

12. The measurement ring of claim 1, wherein the communication module of the measurement ring communicates wirelessly with the communication unit using a standard communication protocol.

13. The measurement ring of claim 1, wherein the communication module comprises a processor, a memory and an antenna.

14. The measurement ring of claim 13, wherein the communication module of the measurement ring is adapted for receiving a command signal from the communication unit for actuating one of the at least one sensor for measuring a particular parameter of insertion of the at least one instrument in the insertion channel.

15. The measurement ring of claim 13, wherein the communication module of the measurement ring transmits the measured parameter when the measured parameter exceeds a predetermined threshold.

16. (canceled)

17. The monitoring apparatus of claim 1, further comprising a display for displaying the received insertion data.

18. The monitoring apparatus of claim 1, wherein the monitoring engine further provides recommendations based on the comparison of the received insertion data with the ranges of acceptable measurements to a medical professional performing insertion of the at least one instrument through the insertion channel.

19. The monitoring apparatus of claim 18, further comprising a display for graphically representing the recommendations.

20. The monitoring apparatus of claim 1, further comprising a database for storing the received insertion data and generated recommendations from the monitoring engine.
